(19) United States
(12) Patent Application Publication
(10) Pub. No.: US 2002/0156437 A1
(43) Pub. Date: Oct. 24, 2002

(54) Removal of targeted proteases with proteinaceous wound dressings containing growth factors

(75) Inventors: Jason P. McDevitt, Alpharetta, GA (US); David John Tyrrell, Appleton, WI (US)

Correspondence Address: Neil C. Jones, Third Floor, Keenan Building, 1330 Lady Street, Columbia, SC, 29201, US

(73) Assignee: Kimberly-Clark Worldwide, Inc., Neenah, WI

(21) Appl. No.: 10/016,849

(22) Filed: Dec. 14, 2001

Related U.S. Application Data
(60) Provisional application No. 60/257,806, filed on Dec. 22, 2000.

(51) Int. Cl. A61F 13/00; A61F 15/00; A61M 35/00; A61M 27/00

(52) U.S. Cl. 604/290; 604/304; 604/543; 602/43; 602/50

(57) ABSTRACT

A wound dressing and method for treating a wound with a wound dressing are provided. In particular, the wound dressing contains a fibrous component, typically made from silk fibers or wool fibers for attracting and capturing proteases from a wound site that may interfere with the healing of the wound, and a growth factor.

The present application claims the benefit of U.S. Provisional Application Serial No. 60/257,806 filed Dec. 22, 2000, which is incorporated herein by reference thereto.

### FIELD OF THE INVENTION

The present invention relates to protein-containing dressings that provide an advanced healing environment for wounds. In particular, the invention is a method of promoting wound healing by selectively removing proteases from the wound environment with protein-containing dressings that contain growth factors and that act as capturing substrates for the targeted proteases.

### BACKGROUND OF THE INVENTION

Normal wound repair occurs in a sequential series of interrelated phases: 1) hemostasis; 2) inflammation; 3) proliferation; and 4) remodeling. Wounding induces blood to coagulate forming a plug to prevent fluid and blood loss. This proteinaceous plug also serves as a provisional matrix for cells to migrate into the wound. In addition, growth factors and chemoattractant agents released from activated platelets in the clot help stimulate new tissue growth. During the inflammation phase, cellular and matrix debris and invading microorganisms are removed by immune cells, in particular, neutrophils and macrophages. This provides a suitable wound environment for the next phase. The proliferation phase encompasses both the synthesis and deposition of new extracellular matrix by the fibroblast and the migration and proliferation of both fibroblast and epidermal cells to heal the injured area. In addition, newly formed blood vessels supply the growing tissue with a needed blood supply.

The final phase in the wound healing sequence, which can last for many years, involves a remodeling of the injured tissue to impart greater tensile strength. See J. M. Davidson, Wound repair. In Inflammation: Basic Principles and Clinical Correlates pp. 809-819 (2d. ed. 1992). The process of wound healing in chronic wounds, however, stagnates at some point during the healing sequence. Usually, the process is impeded some time during the inflammation phase. While wound-care researchers debate the actual cause of such stagnation, many scientists point to the presence of excess proteases as an impediment to wound healing.

Proteases such as plasmin, collagenase, gelatinase, and elastase degrade extracellular matrix proteins that are involved in forming connective tissue scaffolds for skin cell migration and proliferation. Aberrant degradation of extracellular matrix proteins is a consequence of an imbalance between the proteases and their natural inhibitors. If the normal balance between proteases and their inhibitors in a chronic wound environment could be restored, wound healing should be improved.

Neutrophil elastase is highly elevated in non-healing wounds and has been implicated to contribute to the chronic wound state. Nwomeh, Yager, and Cohen, Physiology of the chronic wound, Clinics 25 Plastic Surgery 341-356 (1998). This serine protease has a specificity for peptide bonds adjacent to neutral amino acids. Neutrophil elastase will hydrolyze a wide variety of protein substrates.

A limited number of approaches have been suggested to selectively inactivate and remove such deleterious proteases from wounds. Methods to achieve this process have been proposed, either via removal from the wound site by covalent attachment, high-specificity binding, or gel filtration; use of narrow pore size membranes at the wound site to capture the targeted proteases; or inactivation by addition of selective inhibitors to the wound site.

For example, the inhibition of collagenase has been attempted by companies such as CollaGenex. In particular, CollaGenex has been investigating the potential use of chemically modified tetracyclines, which act as inhibitors of matrix metalloproteinases, for the treatment of wounds such as burns and ulcers. In Metalloproteinase Inhibitors and Wound Healing: A Novel Enhancer of Wound Strength, 124 Surgery 464-70 (1998), Witte et al. described an investigation regarding the role of collagenases in wound healing and concluded that inhibition of metalloproteinase activity could be inhibited by decreasing collagen turnover or increasing collagen maturation and crosslinking.

While the removal of highly substrate-specific collagenases from a wound site may be desirable, a more effective wound healing strategy might entail removing broad-spectrum proteases (i.e., proteases that can tolerate many different substrates) such as elastase. Elastase and other broad-spectrum proteases may also activate latent collagenases in the wound environment that can accelerate extracellular matrix turnover.

Various wound dressings that contain proteins have been utilized. Typically, such dressings have employed silk or wool proteins. Examples of such wound dressings are disclosed in Japanese Patent Nos. JP-11104228 to Tsubouchi et al. and JP-11049659 to Ninakawa et al. The dressings contain an amorphous silk protein—silk fibroin. Silk fibroin is an insoluble protein that is an essential component of raw silk. Although fibroin supports proliferation of human skin cells, the protein, without any accompanying protein-containing fibrous component, can only passively absorb proteases on its surface.

Additionally, wound dressings comprised of wool and treated animal fibers are disclosed in French Patent No. 2,751,870 to Birbeau et al. and European Patent No. 468,797 to Koga et al. In particular Koga et al. disclosed a method of using wool to remove the outer keratin layers of the wound surface. Finally, the removal of matrix metalloproteinases from wound sites by molecular sieves was proposed in British Patent No. GB 2,326,827.

The prior art, however, is deficient in demonstrating the ability of silk and wool non-fibrous proteins to sequester proteases and remove them from non-healing wounds. Protein-containing fibers have not heretofore been employed for removing targeted proteases from wound sites as provided by the present invention and, in particular, have not been provided in conjunction with growth factor therapy as set forth herein.

### SUMMARY OF THE INVENTION

The present invention recognizes and addresses some of the foregoing drawbacks, and deficiencies of prior art constructions and methods.

Wound dressings can absorb or adsorb proteins and other compounds from wound fluid. However, further contact with wound fluid can release these compounds back into the wound fluid in a dynamic equilibrium process. Generally speaking, the present invention is directed to wound dressings comprised of protein-containing fibers and growth factors that selectively sequester targeted proteases from wound sites, effectively removing them from the dynamic equilibrium process, and thereby promoting wound healing. The growth factors accompanying the dressings replace or compliment growth factors that may already be present at the wound site.

Dressings manufactured from the protein-containing fibers may also include various other fibrous components, either simply combined with the protein-containing fibers when the dressings are formed, interwoven with the protein-containing fibers, or coated with various growth-promoting and wound-healing additives such as, for example, chitosan or alginate.

More specifically, the present invention involves treating the wound with a dressing that contains either silk or wool fibers and that further contains one or more growth factors. Such dressings may contain, in addition to silk fibers, either wool fibers, or mixtures of silk and wool fibers, and/or various non-proteinaceous materials.

### DETAILED DESCRIPTION OF A REPRESENTATIVE EMBODIMENT

Reference will now be made in detail to the embodiments of the invention, one or more examples which are set forth below. Each example is provided by way of explanation of the invention, not limitation of the invention. In fact, it will be apparent to those skilled in the art that various modifications and variations can be made in the present invention without departing from the scope or spirit of the invention.

In general, the present invention entails the treatment of wounds with dressings that contain protein-containing fibers and at least one growth factor. Such protein-containing fibers are chosen based on the proteases that are targeted for removal from the wound site. By removing such proteases, wound healing is allowed to proceed more rapidly. Thus, wounds dressed in the inventive protein-containing fibers are expected to accelerate the rate of wound closure.

Dressings manufactured from the protein-containing fibers may also include various other fibrous components, either simply combined with the protein-containing fibers when the dressings are formed, interwoven with the protein-containing fibers, or coated with various growth-promoting and wound healing additives such as, for example, chitosan or alginate.

The inventive wound dressings are more effective when the protein-containing fibers are in the form of a fabric, rather than as a mass of fibers or yarns. Fabrics can be woven, knitted, or nonwoven. The preference for fabrics relative to fibers may be because of a higher ratio of volume to surface area.

In certain embodiments, the inventive wound dressings will employ particularly either wool fibers, silk fibers, or a combination of both wool and silk fibers. In all embodiments, the protein-containing fibers may be combined with various non-proteinaceous materials, including non-protein-containing fibers, to form the inventive wound dressings.

In accordance with the present invention, it has been discovered that protein-containing fibers absorb and remove various proteases from wound sites. It is believed that the protein-containing fibrous substrates employed herein allow a protease to tunnel into the interior of the dressings because the protein fibers, or specific regions thereof, are substrates for the targeted protease. Hence, the protease cuts into the fiber, thereby moving away from the surface and effectively becoming removed from the equilibrium process at the fiber surface. Thus, such deleterious proteases may be permanently and disproportionately removed from the wound site upon changing of such dressings.

Targeted proteases for the present inventive wound dressings include certain collagenases and gelatinases, in particular those from the immune cells in the wound environment, elastase, and plasmin. In certain embodiments, silk or silk-related fibers are employed in the dressings to remove elastase from wound sites. Neutrophil elastase degrades extracellular constituents and may also activate latent proteases in the wound microenvironment. Certain collagenases and gelatinases, particularly from immune cells in the wound environment, are also probable targets because of their elevation in chronic wounds. Nwomeh, Yager, Cohen, Physiology of the chronic wound. 25 Clinics Plastic Surgery 341-56 (1998). In addition, this protein-inventive dressing has the potential of regulating the activity of urokinase plasminogen activator and plasmin that have been implicated to contribute to the chronic wound state. Expression and proteolysis of vascular endothelial growth factor is increased in chronic wounds. Wysocki, Kusakabe, Chang, Tuan, 115 J. Invest. Dermatol. 12-8 (1999). Temporal expression of urokinase plasminogen activator, plasminogen activator inhibitor and gelatinase-B in chronic wound fluid switches from a chronic to an acute wound profile with progression to healing. 7 Wound Repair Regn. 154-65.

In one particular embodiment of the present invention, a fibrous wound dressing comprised solely of protein-containing fibrous materials, in combination with one or more growth factors, is employed. In another particular embodiment, a fibrous wound dressing comprised of both fibrous protein-containing materials/growth factors and non-proteinaceous materials and fibers, such as cotton fiber, is employed.

In an embodiment of the present invention, the wound dressing contains a silk fiber textile material. The dressing may be comprised entirely of fibrous silk or may include other materials such as cotton or non-fibrous proteins. Silk can be processed into a fabric, yarn, or fibers and then formed into such inventive wound dressings by known processes. The texture of the dressing can vary: the silk can be creped or cloqued, or be a georgette material. High quality silk is not required, but may be useful in certain embodiments. In addition, the silk-containing material may be processed in various ways, depending on the end product desired. For example, the silk fiber-containing material may be dyed or otherwise treated with various indicia.

With respect to this particular embodiment, silk has been found particularly effective in selectively removing elastase as well as other broad spectrum proteases from the wound environment. Because neutrophil elastase can contribute to the non-healing or slow-healing of wounds by degrading tissue and growth factors necessary for tissue repair, removal of neutrophil elastase may promote wound healing. Dressings comprised of silk fibers may, in certain circumstances, be preferable to wool, gelatin, and collagen-based fibers in selectively removing elastase from the wound site. In addition, from a practical standpoint, wool may be too hairy; gelatin is not fibrous and therefore will not exhibit the entrapment characteristics of wool and silk fibers; and collagen-based products are relatively expensive.

Silk fibers may also be added to existing wound dressings, interwoven with other textiles, or coated for example with chitosan or alginate or other wound-healing promotion additives. In one particular embodiment, silk may be interwoven with a cotton gauze.

Once the deleterious proteases are removed from the wound site, it would be beneficial to add additional tissue growth factors to promote additional wound healing. Therefore, in the present invention, various growth factor treatments can be included in the fibrous wound dressing to improve the therapeutic efficacy of the dressing.

Such growth factors can, optionally, be applied to the wound as an ointment, lotion, solution, gel, etc., after which the wound is covered with the inventive protein-containing dressing. Alternatively, the growth factors or tissue-growth enhancing compositions can be included as part of the wound dressing itself. Such impregnation of the fibrous dressings or coating of the fibrous dressings with growth factors can allow controlled release of the active growth factors while simultaneously attracting and capturing deleterious proteases such as elastase from the wound site. Also, growth factors and/or cytokines can be attached to the protein fibers of the wound dressing via collagenase, neutrophil elastase, gelatinase, or plasmin -recognized peptide substrates such that upon protease hydrolysis, the growth factor and/or cytokine is released into the wound environment to promote healing.

For example, cytokines, chemokines, and growth factors may be included in the dressing. In further example, platelet-derived growth factor is included in the commercially available REGRANEX®. One could contemplate the use of other growth factors, including vascular endothelial growth factor, transforming growth factor beta, basic fibroblast growth factor, keratinocyte growth factors, epidermal growth factor, and peptides derived from extracellular matrix proteins that include collagens, fibronectin, and vitronectin.

### EXAMPLES

The present invention may be understood by reference to the following Examples, without being limited thereto. The Examples were performed in order to demonstrate the removal of proteases from wounds with protein-containing wound dressings in a simulated environment.

In these Examples, stamped wool circles were employed as models for wound tissue; the added textile materials (e.g., silk yarns) were the model for the wound dressings; and the solution containing the particular enzyme was the model for wound fluids. Wool was chosen to represent the wound tissue because broad-spectrum proteases degrade wool, in an analogous fashion to the degradation of tissue in non-healing wounds by overactive proteases. As a result of proteolytic treatment, peptides and amino acids were released from the wool, causing the wool fabric to lose weight as material was transferred into solution. The addition of textiles to a solution comprised of wool and protease is an effective method to simulate and test the ability of other textiles to absorb proteases that would otherwise degrade wool. The general concept of this basic model is that the dressing will remove overexpressed proteases from the wound environment, thereby allowing the tissue to build and the wound to heal.

In this model, a suitable dressing will protect the stamped wool circles by preserving their weight through removal of the proteases from the equilibrium concentration. To determine whether the dressing was effective in preserving the weight of the wool samples, the initial weights of the wool samples were determined and then the wool samples were added to solution along with any protective dressing. Proteases or other enzymes were then added, and the reaction contents were agitated on a laboratory shaker. The wool samples were then rinsed and dried overnight. Finally, the samples were weighed again to determine the change in weight (Δw). The effectiveness of the protective dressing was determined by comparing the observed weight loss to the average weight loss in the control samples in which no protective dressing was added.

A broad-spectrum bacterial subtilisin protease was used in some examples to test the model system's ability to remove generic broad-spectrum proteases. Porcine pancreatic elastase was also used in some examples to test the efficiency of protein-containing dressings in removing a mammalian elastase from the wound site. Porcine pancreatic elastase shares substantial amino acid homology with human neutrophil elastase and is very similar in mode of action, albeit with some differences in inhibitor sensitivity and relative specificity.

### Examples 1-15

The ability of various textiles to selectively remove proteases from wounds was determined as follows. Two stamped wool flannel circles were prepared. Each circle was two inches in diameter and weighed approximately 0.5 grams (g). In Examples 1-15, the wool samples, along with any textile model wound dressing, were added to 25 milliliters (mL) of 1.5% sodium bicarbonate solution, followed by 25 microliters (μL) of ESPERASE® (a bacterial subtilisin protease obtained from Novo Nordisk Biochem North America Inc.). The wool circles were then shaken in this solution for 8 hours in 1-ounce vials. After drying overnight, the samples were then weighed to determine a change in weight. An average weight was then obtained for the two samples for each Example and the percent of protease removed was determined.

Examples 1-7 were undertaken separately from Examples 8-15. The data within each set of Examples is accurate relative to the other Examples within each set. Because of variations in experimental conditions (e.g., humidity), the absolute values shown in the Examples should not be compared across data sets.

As shown in Tables 1 and 2, the protein fiber-containing dressings, both wool and silk, effectively removed proteases from the model wound fluid, thereby protecting the model wound tissue (wool circles) from proteolytic degradation. In Examples 2, 3, 4, 7, 10, 11, and 12, it was demonstrated that the model wound tissue was protected from protease hydrolysis as the weight of the wool circles was substantially preserved by inclusion of the protein-fiber containing dressing. Protein fabrics (Examples 2 and 7) removed the proteases from equilibrium circulation more effectively than yarns (Examples 3 and 4) on an equivalent weight basis.

In contrast to the Examples utilizing protein fiber-containing model dressings, very little if any preservation of the model wound tissue was observed in Examples that employed non-protein-containing dressings such as a polypropylene SMS nonwoven (Examples 13, 14), cotton gauze (Example 15), cotton twine (Example 5), a paper towel (Example 6), or the control examples in which no dressing was added (The SMS nonwoven is a three-layer laminate having spunbond/meltblown/spunbond layers of synthetic polypropylene).

TABLE 1

| | | ΔW1 | ΔW2 | ΔAvg | |
|---|---|---|---|---|---|
| Example | Textile Added | (mg) | (mg) | (mg) | % Protected |
| 1 | Control | 14 | 15 | 14.5 | — |
| 2 | Silk gauze (133 mg) | 11 | 11.5 | 11.25 | 22.4 |
| 3 | Silk yarn (502 mg) | 12.5 | 12 | 12.25 | 15.5 |
| 4 | Silk/wool yarn | 12 | 11.5 | 11.75 | 19.0 |
| | (721 mg) | | | | |
| 5 | Cotton twine | 13.5 | 13 | 13.25 | 8.6 |
| | (1120 mg) | | | | |
| 6 | Paper Towel | 12.5 | 15 | 13.75 | 5.2 |
| | (186 mg) | | | | |
| 7 | Wool Flannel | 5 | 5 | 5 | 65.5 |
| | (881 mg) | | | | |

TABLE 2

| | | ΔW1 | ΔW2 | ΔAvg | |
|---|---|---|---|---|---|
| Example | Textile Added | (mg) | (mg) | (mg) | % Protected |
| 8 | Control | 19.3 | 16.8 | 18.05 | −3.9 |
| 9 | Control | 17.3 | 16.1 | 16.7 | 3.9 |
| 10 | Wool flannel (432 mg) | 10.4 | 11.7 | 11.05 | 36.4 |
| 11 | Silk gauze (110.7 mg) | 13.4 | 14.1 | 13.75 | 20.9 |
| 12 | Silk gauze (560 mg) | 6.5 | 6.8 | 6.65 | 61.7 |
| 13 | SMS nonwoven | 15.9 | 16.0 | 15.95 | 8.2 |
| | (105 mg) | | | | |
| 14 | SMS nonwoven | 18.3 | 16.0 | 17.15 | 1.3 |
| | (625 mg) | | | | |
| 15 | Cotton gauze | 14.6 | 16.8 | 15.9 | 8.5 |

### Examples 16-22

The ability of a protein-containing dressing of the present invention to selectively remove elastase from wounds was demonstrated in Examples 16-22 as follows. Two stamped circles of wool flannel, two inches in diameter and approximately 0.5 g in weight, along with any model dressings, were added to 25 mL of 1.5% sodium bicarbonate solution. To that solution, 20 μL of a mammalian elastase—(porcine pancreatic elastase from Sigma, EC 3.4.21.36)—was then added. The elastase contained 5.1 mg of protein per mL and 6.3 units of protein per mg where by definition 1 unit of enzyme 1 μmole of the substrate Suc-Ala-Ala-Ala-pNP per minute at pH 8.0 at 25° C. The wool circles and the solution were shaken for 8 hours in 1-ounce vials.

As shown in Table 3, the wool and silk fiber-containing dressings were effective (Examples 18, 19, and 22) in removing elastase from equilibrium circulation, whereas the non-protein dressings (Examples 20 and 21) were relatively ineffective in removing the elastase.

The percentage of weight protection for Examples 18, 19, and 22 was virtually identical. The amounts of the added protein fiber-containing dressing necessary to reach that level of elastase removal, however, were not the same. On a per weight basis, silk gauze (Example 18) was the most effective at removing elastase from equilibrium concentration, followed by wool flannel (Example 19), followed by the blend of silk and wool fibers (Example 22).

TABLE 3

| | | ΔW1 | ΔW2 | ΔAvg | |
|---|---|---|---|---|---|
| Example | Material Added | (mg) | (mg) | (mg) | % Protected |
| 16 | Control | 10.2 | 12.5 | 11.35 | −10% |
| 17 | Control | 9.8 | 8.8 | 9.3 | 10% |
| 18 | Silk gauze | 8.4 | 7.5 | 7.95 | 23 |
| | (174 mg) | | | | |
| 19 | Wool flannel | 8.3 | 7.8 | 8.05 | 22 |
| | (375 mg) | | | | |
| 20 | SMS nonwoven | 10.3 | 10.0 | 10.15 | 2 |
| | (251 mg) | | | | |
| 21 | Polypropylene/ | 12.0 | 14.5 | 13.25 | −28 |
| | cellulose | | | | |
| | Conform nonwoven | | | | |
| | (451 mg) | | | | |
| 22 | Bombyx silk/merino | 7.9 | 8.2 | 8.05 | 22 |
| | wool | | | | |
| | fibers (788 mg) | | | | |

### Examples 23-30

The ability of dressings containing protein fibers as compared to dressings containing protein fabrics to selectively remove elastase from wounds was determined in Examples 23-30 as follows. Two stamped circles of wool flannel, two inches in diameter and approximately 0.5 g in weight, were added, along with any model wound dressings, to 25 mL of 1.5% sodium bicarbonate solution. To this solution, 20 μL of a mammalian elastase—(porcine pancreatic elastase from Sigma, EC 3.4.21.36)—was then added. The elastase contained 5.1 mg of protein per mL and 6.3 units of protein per mg where by definition 1 unit of enzyme hydrolyzes 1 μmole of the substrate Suc-Ala-Ala-Ala-pNP. The wool circles and the solution were shaken for 24 hours in 1-ounce vials.

The results are provided in Table 4. Silk gauze was superior to wool or silk fibers, or wool flannel, providing superior containment of the elastase. As modeled in Example 27, in which the silk gauze was removed after 5 hours, changing the wound dressing provided the most advantageous results because the captured deleterious proteases are permanently and irreversibly removed from the wound site. The various model dressings that serve as substrates for the proteases can be effective in sequestering the proteases for moderate time periods but ultimately, the proteases can tunnel back out of or through the interior of the fibrous dressing and return to the surface, where the dynamic equilibrium process with the simulated wound fluid can return them into solution to degrade other substrates such as the model wound tissue.

TABLE 4

| | | ΔW1 | ΔW2 | ΔAvg | |
|---|---|---|---|---|---|
| Example | Materials Added | (mg) | (mg) | (mg) | % Protected |
| 23 | Control | 29.3 | 28.4 | 28.9 | 2 |
| 24 | Control | 30.1 | 29.6 | 29.9 | −2 |
| 25 | Silk gauze | 16.9 | 16.9 | 16.9 | 43 |
| | (174 mg) | | | | |
| 26 | Silk gauze | 17.4 | 21.4 | 19.4 | 34 |
| | (588 mg) | | | | |
| 27 | Silk gauze | 9.2 | 10.2 | 9.7 | 67 |
| | (541 mg), removed | | | | |
| | after 5 hours | | | | |
| 28 | Wool flannel | 27.4 | 26.8 | 27.1 | 8 |
| | (455 mg) | | | | |
| 29 | Merino wool fibers | 26.3 | 32.7 | 29.5 | 0 |
| | (788 mg) | | | | |
| 30 | Silk fibers | 30.5 | 35.0 | 32.8 | −12 |
| | (654.5 mg) | | | | |

### Examples 31-33

The ability of dressings containing protein fibers to selectively remove enzymes from a model wound solution is demonstrated by comparing the previous Examples, in which elastase activity was removed from solution, with the present set of Examples (31-33), in which horseradish peroxidase activity was not reduced in the presence of a wool fabric model dressing. An aqueous solution containing water (5 mL) and a solution of horseradish peroxidase enzyme (type 6A, EC 1.11.1.7, obtained from Sigma; enzyme solution was diluted to 55 units per mL, wherein by definition one unit will oxidize 1 μmole 2,2′-azino-bis(3-ethylbenzthiazoline-6-sulfonic acid) per minute at 25° C. at pH 5.0) was shaken in the presence or absence of wool flannel (200 mg). The amount of horseradish peroxidase was varied in order to demonstrate that removal of the protease could be detected by the assay. After shaking one hour, 50 μL of the solution was removed and added to 3 mL water, to which was added 50 μL of a substrate for the horseradish peroxidase enzyme (TMB Microwell Peroxidase substrate, available from Kirkegaard & Perry Laboratories). The substrate was colorless, but turned blue-green upon reaction with the enzyme. After two minutes, the absorbency at 445 nm was read on a spectrophotometer.

As is apparent from the data in Table 5, wool flannel was not effective in sequestering the horseradish peroxidase enzyme. Significant removal of the horseradish peroxidase from solution by wool would have yielded a reduced observed enzyme activity (as was the case when the enzyme concentration was intentionally halved in Example 33). The observed activity in Example 32 was statistically indistinguishable from that in Example 31 (data averaged over 3 trials), indicating that the wool flannel did not significantly reduce the solution concentration of horseradish peroxidase.

TABLE 5

| | Horseradish | | |
|---|---|---|---|
| | Peroxidase | Wool Flannel | |
| Example | (μL) | (mg) | Absorbance |
| 31 | 20 | 0 | 0.0360 |
| 32 | 20 | 200 | 0.0357 |
| 33 | 10 | 0 | 0.0220 |

To any of the suitable protein-containing wound dressings, various growth factors may be added to either replace growth factors that are removed from or destroyed at the wound site or to compliment the growth factors that are present at the site. The various growth factors suitable for adding to the proteinaceous wound dressings include any of the factors described above, as well as other known or yet-to-be-developed growth factors.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims. Therefore, the spirit and scope of the appended claims should not be limited to the description of the preferred versions contained therein.

What is claimed is:

1. A method for treating a wound by removing a protease from the site of the wound, said method comprising the steps of:
(a) selecting a protein-containing fibrous component capable of removing a protease;
(b) forming a dressing from said protein-containing fibrous component;
(c) selecting at least one protein from the group consisting of growth factors, cytokines, and chemokines for application to said wound site;
(d) applying said dressing and said protein to said wound and allowing at least a portion of said protease found at said wound site to be attracted to and entrapped by said protein-containing fibrous component; and
(e) removing said dressing from said wound so that at least a portion of said protease is removed from said wound site.

2. The method of claim 1 wherein said protein-containing fibrous component comprises silk fiber.

3. The method of claim 1 wherein said protein-containing fibrous component comprises wool fiber.

4. The method of claim 1 wherein said protein-containing fibrous component comprises a protein-containing fabric.

5. The method of claim 4 wherein said protein-containing fibrous component comprises silk gauze.

6. The method of claim 1 wherein said dressing is formed from a non-protein-containing material in addition to the protein-containing fibrous component.

7. The method of claim 6 wherein said non-protein-containing material comprises cotton fibers.

8. The method of claim 7 wherein said cotton fibers are interwoven with said protein-containing fibrous component.

9. The method of claim 8 wherein said protein-containing fibrous component comprises silk fibers.

10. The method of claim 1 wherein said protease comprises elastase.

11. The method of claim 1 wherein said protease comprises neutrophil elastase.

12. The method of claim 1 wherein said protease comprises gelatinase.

13. The method of claim 1 wherein said protease comprises gelatinase B (MMP-9).

14. The method of claim 1 wherein said protease comprises plasmin.

15. The method of claim 1 wherein said protein is applied to said wound site as a component separate from said dressing.

16. The method of claim 15 wherein said protein is applied to said wound site in the form of an ointment, lotion, solution, or gel.

17. The method of claim 1 wherein said protein is included as part of the wound dressing itself.

18. The method of claim 1 wherein said growth factor is chosen from the group consisting of platelet-derived growth factors, vascular endothelial growth factors, transforming growth factors, fibroblast growth factors, and epidermal growth factors.

19. A method for treating a wound by removing a protease from the site of the wound, said method comprising the steps of:
(a) applying a wound dressing and at least one growth factor to said wound site wherein said wound dressing comprises a protein-containing fibrous component capable of removing said protease; and
(b) allowing said wound dressing to withdraw and entrap said protease so that healing of said wound is promoted.

20. The method of claim 19 wherein said growth factor is applied to said wound site as a component separate from said dressing.

21. The method of claim 20 wherein said growth factor is applied to said wound site in the form of an ointment, lotion, solution, or gel.

22. The method of claim 19 wherein said growth factor is included as part of the wound dressing itself.

23. The method of claim 19 wherein said growth factor is chosen from the group consisting of platelet-derived growth factors, vascular endothelial growth factors, transforming growth factors, fibroblast growth factors, and epidermal growth factors.

24. A wound dressing for removing a protease from the site of the wound and supplying a growth factor to said wound site, said dressing comprising:
(a) a protein-containing fibrous component; and
(b) at least one growth factor
wherein a protease found at said wound site may be attracted to and entrapped by said protein-containing fibrous component.

25. The wound dressing of claim 24 wherein said protein-containing fibrous component comprises silk fiber.

26. The wound dressing of claim 24 wherein said protein-containing fibrous component comprises wool fiber.

27. The wound dressing of claim 24 wherein said protein-containing fibrous component comprises a protein-containing fabric.

28. The wound dressing of claim 27 wherein said protein-containing fibrous component comprises silk gauze.

29. The wound dressing of claim 24 wherein said dressing further comprises a non-protein-containing material in addition to the protein-containing fibrous component.

30. The wound dressing of claim 29 wherein said non-protein-containing material comprises cotton fibers.

31. The wound dressing of claim 30 wherein said cotton fibers are interwoven with said protein-containing fibrous component.

32. The wound dressing of claim 24 wherein said growth factor is chosen from the group consisting of platelet-derived growth factors, vascular endothelial growth factors, transforming growth factors, fibroblast growth factors, and epidermal growth factors.
